(19) United States
(12) Patent Application Publication
(10) Pub. No.: US 2025/0121200 A1
(43) Pub. Date: Apr. 17, 2025

(54) HERMETIC CONNECTOR STACKS FOR AN ELECTRICAL STIMULATION SYSTEM

(71) Applicant: Boston Scientific Neuromodulation Corporation, Valencia, CA (US)

(72) Inventors: Thomas Paul Lopez, Sunland, CA (US); Samuel N Amundson, Canyon Country, CA (US)

(73) Assignee: Boston Scientific Neuromodulation Corporation, Valencia, CA (US)

(21) Appl. No.: 18/911,991

(22) Filed: Oct. 10, 2024

Related U.S. Application Data
(60) Provisional application No. 63/543,804, filed on Oct. 12, 2023.

(51) Int. Cl. A61N 1/375; H01R 24/58

(52) U.S. Cl. CPC A61N 1/3752; H01R 24/58; H01R 2201/12

(57) ABSTRACT

A connector stack includes a plurality of conductive rings and a plurality of insulator rings. The plurality of insulator rings separating the conductive rings from each other. The plurality of conductive rings and the plurality of insulator rings form the connector stack, and define a lumen therethrough. The plurality of conductive rings and the plurality of insulator rings are brazed together such that the connector stack is a hermetically sealed stack. The conductive rings have one or more of a counterbore design, a conical design, or a D-flat profile. The insulator rings may be disk shaped or may have a shape similar to the conductive rings.

### CROSS-REFERENCE TO RELATED APPLICATION

This application claims the benefit of U.S. Provisional Patent Application Ser. No. 63/543,804, filed Oct. 12, 2023, which is incorporated herein by reference.

### TECHNICAL FIELD

The present disclosure relates generally to implantable medical devices, and more particularly to hermetic connector stacks for an implantable electrical stimulation system.

### BACKGROUND

Implantable electrical stimulation systems are commonly used to provide therapy for a variety of diseases and disorders. For example, an implantable medical device may deliver neurostimulation therapy to a patient. For example, spinal cord stimulation systems may be used as a therapeutic modality for the treatment of chronic pain. Deep brain stimulation systems provide electrical signals to the brain to treat, for example, tremor associated with Parkinson's Disease. In some examples, an implantable medical device may be configured to deliver pacing and/or defibrillation therapy to a patient.

Many implantable medical device systems include an implantable canister and a lead. The attachment of the lead to the implantable canister is achieved in many cases with a header secured the canister having a port for receiving a proximal end of the lead, such that the distal end of the lead can extend from the canister to a therapy target, such as the heart, spinal cord, brain, or other location depending on the device. The lead distal end may have several electrodes, each with a corresponding contact at the proximal end of the lead. The header will include a plurality of electrical connectors allowing each lead contact to be separately electrically coupled to operational circuitry inside the canister. The electrical connectors in the header may be held in a connector stack having conductive electrical connectors separated by dielectric pieces. The connector stacks in some conventional designs is sensitive to side loading, and many are not well suited to direct surface mount on a circuit board, as may be useful for miniaturized devices or “microstimulators. New and alternative designs are desired.

### BRIEF SUMMARY

The present disclosure relates generally to implantable medical devices, and more particularly to hermetic connector stacks for an implantable electrical stimulation system. An example connector stack may include a plurality of conductive rings, and a plurality of insulator rings, using a chevron cross sectional design, providing added resistance to side loading. Another example connector stack may include a plurality of conductive rings and a plurality of insulator rings, using a counterbore design, again providing added resistance to side loading. Still another example connector stack may use a D-flat design, facilitating placement on a circuit board. A microstimulator may include any of these designs for a connector stack, and may further include a brazed, soldered, or welded-in-place lead affixed within the bore of the connector stack.

A first illustrative and non-limiting example takes the form of a connector stack comprising: a plurality of conductive rings; and a plurality of insulator rings, the plurality of insulator rings separating the conductive rings from each other, the plurality of conductive rings and the plurality of insulator rings forming the connector stack defining a lumen therethrough; wherein the conductive rings comprise means for resisting a lateral force applied to the connector stack.

Additionally or alternatively, the means for resisting includes a first boss on each of the conductive rings. Additionally or alternatively, the insulator rings each include a second boss and each of the plurality of conductive rings and each of the plurality of insulator rings are configured to stack together thereby forming overlapping counterbore joints. Additionally or alternatively, the counterbore joints overlap within a range of about 20 to 50 percent. Additionally or alternatively, the counterbore joints provide structural support against lateral displacement in the connector stack. Additionally or alternatively, the means for resisting includes a conical shape of each of the plurality of conductive rings. Additionally or alternatively, each of the plurality of insulator rings include a conical shape, and each of the plurality of conductive rings and each of the plurality of insulator rings are configured to stack together thereby forming chevron-shaped joints. Additionally or alternatively, the chevron-shaped joints provide structural support against lateral displacement in the connector stack. Additionally or alternatively, the plurality of conductive rings and the plurality of insulator rings are self-centering when stacked together.

Another illustrative and non-limiting example takes the form of a connector stack, comprising: a plurality of conductive rings; a plurality of insulator rings separating the conductive rings from each other, the plurality of conductive rings and the plurality of insulator rings forming the connector stack defining a lumen therethrough; and a braze material between adjacent ones of the conductive rings and insulator rings; wherein the plurality of conductive rings and the plurality of insulator rings are brazed together such that the connector stack is a hermetically sealed stack; and wherein the connector stack includes a D-flat profile formed by an outer profile of the conductive rings having a D-flat shape.

Additionally or alternatively, the connector stack includes a D-flat surface mount coupled to a proximal end or a distal end of the connector stack. Additionally or alternatively, the connector stack may include a braze material disposed between adjacent ones of each of the plurality of conductive rings and each of the plurality of insulator rings, wherein the plurality of conductive rings and the plurality of insulator rings are brazed together to form a hermetic conductor stack. Additionally or alternatively, an internal diameter of the plurality of conductive rings and an internal diameter of the plurality of insulator rings are the same.

Another illustrative and non-limiting example takes the form of a connector stack, comprising: a plurality of conductive rings; and a plurality of insulator rings, the plurality of insulator rings separating the conductive rings from each other, the plurality of conductive rings and the plurality of insulator rings forming the connector stack defining a lumen therethrough; wherein each of the plurality of conductive rings each including a first boss.

Additionally or alternatively the connector stack comprises a braze material disposed between adjacent ones of each of the plurality of conductive rings and each of the plurality of insulator rings, wherein the plurality of conductive rings and the plurality of insulator rings are brazed together. Additionally or alternatively each of the plurality of insulator rings includes a second boss, and each of the plurality of conductive rings and each of the plurality of insulator rings are configured to stack together thereby forming overlapping counterbore joints. Additionally or alternatively the counterbore joints overlap within a range of about 20 to 50 percent. Additionally or alternatively the counterbore joints provide structural support against lateral displacement of the connector stack.

Another illustrative and non-limiting example takes the form of a connector stack, comprising: a plurality of conductive rings; a plurality of insulator rings separating the conductive rings from each other, the plurality of conductive rings and the plurality of insulator rings forming the connector stack defining a lumen therethrough; and a braze material between adjacent ones of the conductive rings and insulator rings; wherein the plurality of conductive rings and the plurality of insulator rings are brazed together such that the connector stack is a hermetically sealed stack; and wherein the connector stack includes a D-flat profile formed by an outer profile of the conductive rings having a D-flat shape.

Additionally or alternatively, the plurality of insulator rings are ceramic. Additionally or alternatively, the connector stack is brazed together when the braze material flows via capillary action to a braze joint between adjacent ones of the conductive rings and insulator rings. Additionally or alternatively, the connector stack includes a D-flat surface mount coupled to a proximal end or a distal end of the connector stack.

Additionally or alternatively, each of the plurality of conductive rings include a conical shape. Additionally or alternatively, each of the plurality of insulator rings include a conical shape, and each of the plurality of conductive rings and each of the plurality of insulator rings are configured to stack together thereby forming chevron-shaped joints.

Additionally or alternatively, the plurality of conductive rings and the plurality of insulator rings are self-centering when stacked together.

Additionally or alternatively, each of the plurality of conductive rings includes a first boss. Additionally or alternatively, each of the plurality of insulator rings includes a second boss, and each of the plurality of conductive rings and each of the insulator rings are configured to stack together thereby forming overlapping counterbore joints. Additionally or alternatively, the counterbore joints overlap within a range of about 20 to 50 percent.

Another illustrative and non-limiting example takes the form of a connector stack, comprising: a plurality of conductive rings; and a plurality of insulator rings, the plurality of insulator rings separating the conductive rings from each other, the plurality of conductive rings and the plurality of insulator rings forming the connector stack and defining a lumen therethrough; wherein each of the plurality of conductive rings each include a conical shape.

Additionally or alternatively, each of the plurality of insulator rings include a conical shape, and each of the plurality of conductive rings and each of the plurality of insulator rings are configured to stack together thereby forming chevron-shaped joints. Additionally or alternatively, the connector stack also includes a braze material disposed between adjacent ones of each of the plurality of conductive rings and each of the plurality of insulator rings, wherein the plurality of conductive rings and the plurality of insulator rings are brazed together.

Another illustrative and non-limiting example takes the form of an implantable medical device comprising a port for receiving an implantable lead, the port comprising a connector stack as in any of the preceding examples. Another illustrative and non-limiting example takes the form of a microstimulator comprising a connector stack as in any of the preceding examples, and a lead permanently affixed within the bore of the connector stack.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a prior art example of an implantable medical device;

FIG. 2A is a schematic perspective view of an illustrative conductive ring;

FIG. 2B is a perspective view of a connector stack, including a ring as in FIG. 2A;

FIG. 2C is an exploded view of the illustrative connector stack as in FIG. 2B;

FIG. 2D is a cross-sectional view of the illustrative connector stack as in FIG. 2B;

FIG. 3A is a perspective view of an illustrative conductive ring;

FIG. 3B is a perspective view of a connector stack, including a ring as in FIG. 3A;

FIG. 3C is an exploded view of the illustrative connector stack as in FIG. 3B;

FIG. 3D is a cross-sectional view of the illustrative connector stack as in FIG. 3B;

FIG. 4A is a perspective view of an illustrative conductive ring;

FIG. 4B is a perspective view of a connector stack, including a ring as in FIG. 4A;

FIG. 4C is an exploded view of the illustrative connector stack as in FIG. 4B;

FIG. 4D is a sectional view of the illustrative connector stack as in FIG. 4B; and

FIG. 5 is a schematic, perspective view of a portion of an elongated member disposed in an illustrative connector stack, according to the disclosure.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit of the disclosure.

### DETAILED DESCRIPTION

For the following defined terms, these definitions shall be applied, unless a different definition is given in the claims or elsewhere in this specification. All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure. The recitation of numerical ranges by endpoints includes all numbers within that range (e.g., 1 to 5 includes, 1, 1.5, 2, 2.75, 3, 3.80, 4, and 5). As used in this specification and the claims, the singular forms “a”, “an”, and “the” include plural referents unless the context clearly dictates otherwise. As used in this specification and the appended claims, the term “or” is generally employed in its sense including “and/or” unless the content clearly dictates otherwise.

It is noted that references in this specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the claims.

Implantable electrical stimulation systems are commonly used to provide therapy for a variety of diseases and disorders. For example, an implantable medical device may deliver neurostimulation therapy to a patient. For example, spinal cord stimulation systems may be used as a therapeutic modality for the treatment of chronic pain. In some examples, an implantable medical device may be configured to deliver pacing and/or defibrillation therapy to a patient. Such implantable medical devices may treat patients suffering from various heart conditions that may result in a reduced ability of the heart to deliver sufficient amounts of blood to a patient's body. In some cases, heart conditions may lead to rapid, irregular, and/or inefficient heart contractions. To help alleviate some of these conditions, various devices (e.g., pacemakers, defibrillators, etc.) are often implanted into a patient's body.

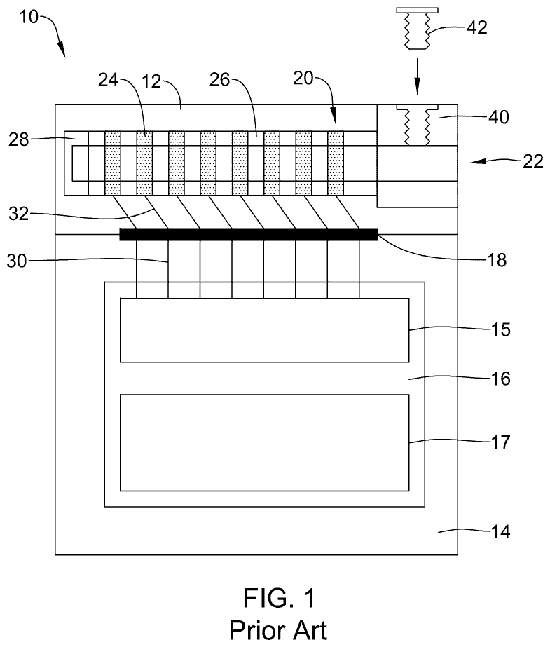

FIG. 1 is a prior art example of a schematic cross-sectional side view of an implantable medical device (IMD) 10 suitable for receiving a lead (e.g., a lead, a lead extension, or other intermediary device). The IMD 10 includes a header 12 disposed along an outer surface of a sealed housing 14 that contains an electronic subassembly 16 with a pulse generator 15 and, optionally, a power supply 17. The pulse generator 15 may comprise, for example, a microcontroller or other control circuitry (logic circuitry, state machine), optionally an associated memory for storing operational data (usage, programmable settings, etc.) as well as instructions readable and executable by the control circuitry for operating the implantable system. Communication circuitry for telemetry (inductive, Bluetooth, Medradio, or other communications whether RF or not) may be included on the electronic subassembly 16, as well as output circuitry such as one or a plurality of voltage sources and/or current sources and associated timing circuitry for therapy outputs. Some examples, both prior and planned future versions, of output circuits, timing circuits and other control circuitry can be found in U.S. Pat. No. 10,716,932, the disclosure of which is incorporated herein by reference. As noted, a permanent power supply 17 can be omitted in some examples, in which case a receiving circuit (such as an inductor or antenna) may receive transmitted power from an external device, and uses, for example, a rectifier and a capacitor for temporary power storage. The power supply 17 may instead be a battery, which can be rechargeable or not rechargeable, as desired. If a rechargeable battery is used at power supply 17, then a recharging circuit may be provided including an inductor or other power receiving element to allow transcutaneous recharging while the IMD 10 is implanted.

A connector stack assembly 20 is disposed in the header 12. The connector stack assembly 20 is configured to receive an elongated device (e.g., a lead, a lead extension, or other intermediary device). The connector stack assembly 20 defines a connector lumen 22 configured to receive a proximal portion of the elongated member. An array of conductive rings, including conductive ring 24, is arranged along the connector lumen 22 and configured to electrically couple with terminals of the elongated member when the proximal portion of the elongated member is received by the connector stack assembly 20. The conductive rings 24 can be electrically isolated from one another by electrically-nonconductive spacers, such as insulator ring 26. The connector stack may also include an end stop 28 to promote alignment of the elongated-member terminals with the conductive rings.

Feedthrough interconnects, such as feedthrough interconnect 30, are electrically coupled to the electronic subassembly 16 and extend within the scaled housing 14 to a feedthrough interface 18 disposed along an interface between the header 12 and the sealed housing 14. The conductive rings 24 are electrically coupled to interconnect conductors, such as interconnect wire 32, that extend along the header 12 and electrically coupled the conductive rings 24 so the feedthrough interconnects at the feedthrough interface 18. In some cases, the header 12 is positioned over the feedthrough interface 18.

The connector stack assembly 20 may include a retention assembly to facilitate retention of the proximal portion of the elongated member when the proximal portion of the elongated member is received by the IMD 10. In an embodiment, the retention assembly includes a retention block 40. The retention block 40 is positioned to align with a retention sleeve of the elongated member when the elongated member is received by the connector stack assembly 20. The retention assembly may have a retaining member (e.g., a set screw, a pin, or the like) 42 for pressing the retention sleeve of the elongated member against the retention block to retain the inserted elongated member within the connector stack assembly 20.

Because the conductive rings 24 and interleaved insulator rings 26 are each disk-like or planar, there is little structural resistance in the conductor stack assembly to lateral forces. This means that the assembled conductor stack can easily be misaligned during assembly processes. The assembly may be, for example, pieced together over a core member that maintains each part in close contact along the central bore, holding alignment. The assembly can then be brazed together, attaching the interconnect wires 32 while also attaching each conductive ring 24 to neighboring insulator rings 26. The core member must, at some point, be removed, after which lateral forces can easily disrupt center bore alignment. In addition, center bore alignment is reliant on manufacturing tolerances of each piece of the assembly, which is a challenge if the insulator rings 26 are made of ceramic or other material that is not easily machined. Alternative designs are desired.

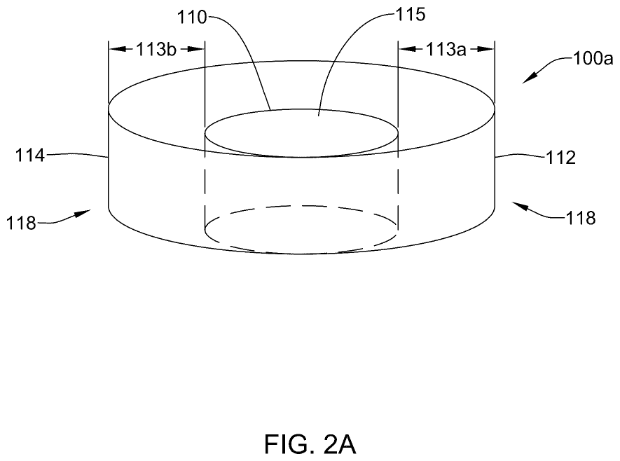

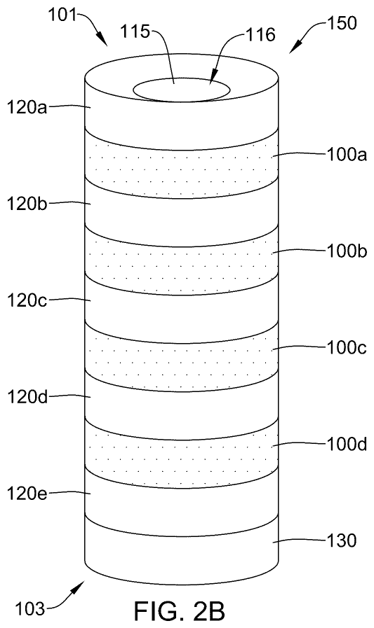

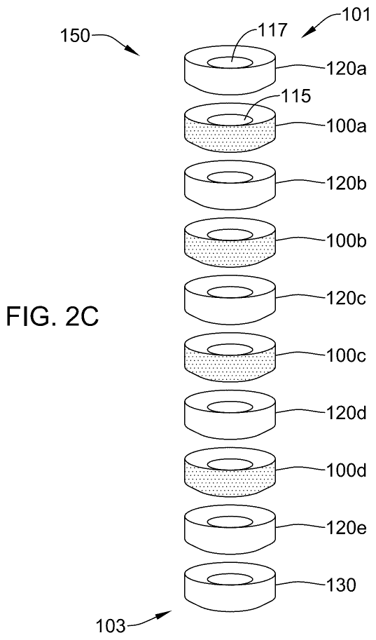

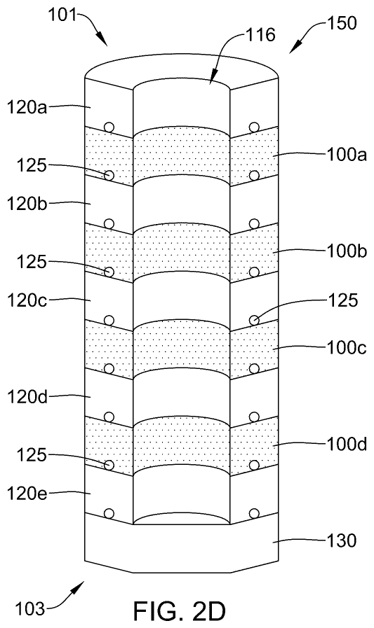

FIGS. 2A to 2D illustrate a connector stack 150, and the parts thereof. For example, FIG. 2A is a schematic perspective view of an illustrative conductive ring 100a. FIG. 2B is a schematic perspective view of an illustrative connector stack 150, including the conductive ring 100a as in FIG. 2A. FIG. 2C is a schematic, perspective exploded view of the illustrative connector stack 150 as in FIG. 2B, and FIG. 2D is a schematic, longitudinal cross-sectional view of the illustrative connector stack 150 as in FIG. 2B. The connector stack 150 may be configured to be used with the IMD 10 illustrated in FIG. 1, or the like. In such cases, the connector stack 150 may replace the connector stack assembly 20.

FIG. 2A illustrates a conductive ring 100a. The conductive ring 100a may be a section of the connector stack 150. The conductive ring 100a may be formed from an electrically conductive material, such as, for example, titanium, stainless steel (MP35N, for example), or other biocompatible and conductive metals. The conductive ring 100a may be formed via casting, working, machining, or the like, and then ground and/or polished as needed. The conductive ring 100a may include a conical shape that may include a first portion 110 having an inner, central bore 115, and a second portion 118 defined by a first surface 112 extending at a first angle 113a relative to the cross-section of the bore, and a second surface 114 extending at a second angle 113b relative to the first portion 110. The first angle 113a and the second angle 113b may fall within in a range of about 10 to about 45 degrees. In some cases, an angle of about 15 to 30 degrees may be preferred. In some cases, as shown in FIGS. 2A to 2D, the first angle 113a and the second angle 113b may be equivalent. However, in some cases, it may be contemplated that the first angle 113a may be greater than the second angle 113b, and/or in some cases, the second angle 113b may be greater than the first angle 113a. These are just examples.

The conductive ring 100a may be one of a plurality of conductive rings 100a, 100b, 100c, 100d, generally referred to herein as conductive rings 100. As shown in FIG. 2B, the conductive rings 100 may be interleaved with sections of electrically nonconductive material and fixedly attached together. In some cases, the interconnected sections of material are stacked along a longitudinal length of the connector stack 150. The connector stack 150 may be configured to receive an elongated device (e.g., a lead, a lead extension, or other intermediary device). In some case, the interconnected sections of material are arranged in an alternating conductive-nonconductive configuration. In some cases, the interconnected sections of material are formed as alternating rings of material. For example, the connector stack 150 may be formed from alternating conductive rings 100, and a plurality of insulator rings 120a, 120b, 120c, 120d, 120e, generally referred to herein as insulator rings 120. The conductive rings 100 may be arranged such that each of the plurality of conductive rings 100 are separated from another one of the plurality of conductive rings 100 by at least one insulator ring 120 in either direction along the longitudinal length of the connector stack 150.

The insulator rings 120 may be formed from a nonconductive material such as, for example, glass, ceramic, or the like. The insulator rings 120 may be formed via sintering, molding, or the like. The insulator rings 120 may include a size and shape similar to that of the conductive rings 100. For example, the insulator rings 120 may include a conical shape that may include a first portion having an inner, central bore 117, and a second portion defined by a first surface extending at a first angle relative to the first portion, and a second surface extending at a second angle relative to the first portion. The first angle and the second angle may fall within in a range of about 10 to about 45 degrees. In some cases, an angle of about 15 to 30 degrees may be preferred. In some cases, an internal diameter of the conductive rings 100 and an internal diameter of the insulator rings 120 may be the same. In some cases, the internal diameter of the conductive rings 100 and an internal diameter of the insulator rings 120 may not be the same. In some cases, the conductive rings 100 and the insulator rings 120 may be stacked using a core wire to hold the rings 100, 120 concentric, therefore the central apertures 115 and 117 align to collectively form a connector lumen 116 that is open at a first end 101 off the connector stack 150. In some cases, the conductive rings 100 and the insulator rings 120 are self-centering when stacked together to form the connector stack 150. When the conductive rings 100 and the insulator rings 120 are stacked and interleaved, the pairs of adjacent rings 100, 120 form a chevron-shaped joint, as seen in FIG. 2D.

In some cases, the interconnected conductive rings 100 and insulator rings 120 may be secured together via brazing. For example, as shown in FIG. 2D, a braze material 126 is positioned between adjacent conductive rings 100 and insulator rings 120. The braze material 126 may be formed from, for example, gold, aluminum, copper, silver, magnesium, or the like. The braze material 126 may be positioned within the connector stack 150 between the conductive rings 100 and the insulator rings 120 as they are stacked. The braze material 126 may come in the form of a thin washer. After the conductive rings 100 and the insulator rings 120 are stacked, the entire connector stack 150 is heated to its brazing temperature such that the braze material 126 liquefies, and the braze material 126 flows via capillary action within the spaces between the conductive rings 100 and the insulator rings 120, thereby bonding the rings 100, 120 together at a braze joint 125. The chevron-shaped joints of the connector stack 150 allow the braze joint(s) 125 to be positioned at a non-perpendicular angle to the longitudinal axis of the connector stack 150. This reduces the amount of force that the braze joint 125 has to withstand under shear (sideways) strain. That is, the conductive ring shape with the conical portions and resulting chevron-shaped joints provide structure that will resist lateral forces. In some cases, the conductive rings 100 and insulator rings 120 may be secured together using any other suitable technique including, for example, 3D printing, co-firing (low or high temperature), or the like.

In some cases, an optional end piece 130 may be disposed along a second end 103 of the connector stack 150. The end piece 130 functions to close the second end 103 of the connector stack 150. In some cases, the end piece 130 is formed from an electrically nonconductive material. In some cases, the connector stack 150 including the end piece 130 may be interconnected to form a hermetic seal. An optional flange (not shown) can be coupled to the first end 101 of the connector stack 150 to facilitate insertion of an elongated member into the connector lumen 116, although this is not needed. A set screw assembly, spring, clip, or other securing element for holding an elongated member in the connector lumen 116 may be provided on either end of the connector stack.

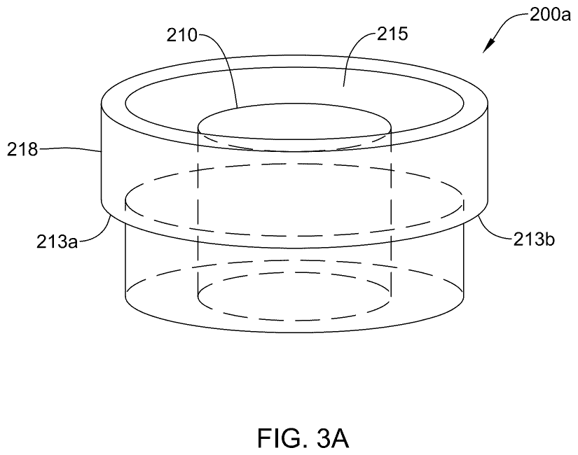

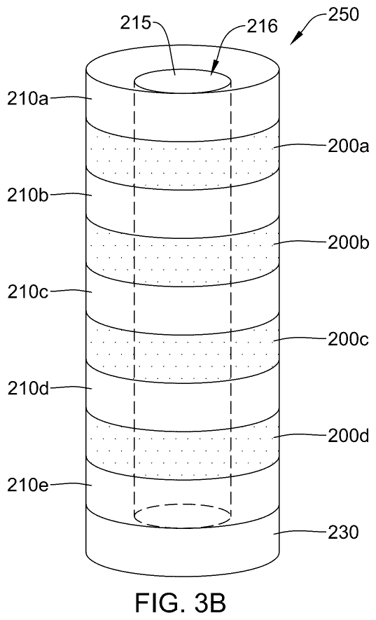

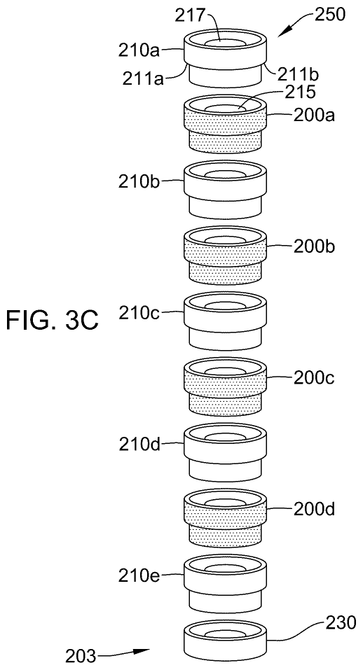

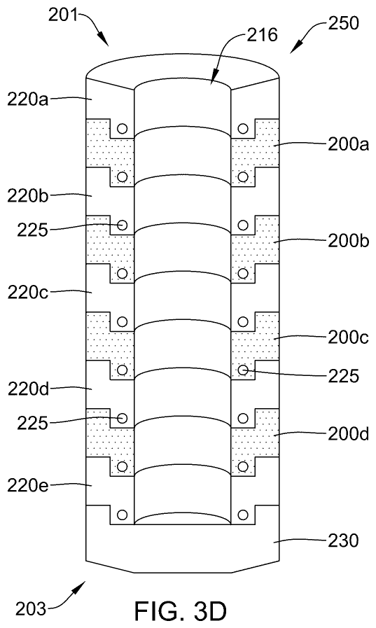

FIGS. 3A to 3D illustrate another connector stack 250, and the parts thereof. For example, FIG. 3A is a schematic perspective view of an illustrative conductive ring 200a. FIG. 3B is a schematic perspective view of an illustrative connector stack 250, including the conductive ring 200a as in FIG. 3A. FIG. 3C is a schematic, perspective exploded view of the illustrative connector stack 250 as in FIG. 3B, and FIG. 3D is a schematic, longitudinal cross-sectional view of the illustrative connector stack 250 as in FIG. 3B. The connector stack 250 may be configured to be used with the IMD 10 illustrated in FIG. 1, or the like. In such cases, the connector stack 250 may replace the connector stack assembly 20.

FIG. 3A illustrates a conductive ring 200a. The conductive ring 200a may be a section of the connector stack 250. The conductive ring 200a may be formed from an electrically conductive material, such as, for example, titanium, stainless steel (MP35N, for example), or other biocompatible conductive material. The conductive ring 200a may be formed via casting, working, machining, or the like, and then ground and/or polished as needed. The conductive ring 200a may include a shape that may include a first portion 210 having a flat disk shape with an inner, central aperture 215, and a second portion 218 including a first boss 213a and a second boss 213b. In some cases, as shown in FIGS. 3A to 3D, the first boss 213a and the second boss 213b may extend away from a longitudinal axis of the central aperture 215. In some cases, as shown in FIGS. 3A to 3D, the first boss 213a and the second boss 213b may include the same size. In some cases, the first boss 213a may be greater than the second boss 213b, and in some cases, the second boss 213b may be greater than the first boss 213a. These are just examples.

The conductive ring 200a may be one of a plurality of conductive rings 200a, 200b, 200c, 200d, generally referred to herein as conductive rings 200. As shown in FIG. 3B, the conductive rings 200 may be interleaved with sections of electrically nonconductive material fixedly attached together. In some cases, the interleaved sections of material are stacked along a longitudinal length of the connector stack 250. The connector stack 250 may be configured to receive an elongated device (e.g., a lead, a lead extension, or other intermediary device). In some case, the interconnected sections of material are arranged in an alternating conductive-nonconductive configuration. In some cases, the interconnected sections of material are formed as alternating rings of material. For example, the connector stack 250 may be formed from alternating conductive rings 200, and a plurality of insulator rings 220a, 220b, 220c, 220d, 220c, generally referred to herein as insulator rings 220. The conductive rings 200 may be arranged such that each of the plurality of conductive rings 200 are separated from another one of the plurality of conductive rings 200 by at least one insulator ring 220 in either direction along the longitudinal length of the connector stack 250.

The insulator rings 220 may be formed from a nonconductive material such as, for example, glass, ceramic, or the like. The insulator rings 220 may be formed via sintering, molding, or the like. The insulator rings 220 may include a size and shape similar to that of the conductive rings 200. For example, the insulator rings 220 may include a shape that may include a first portion having a flat disk shape with an inner, central aperture 217, and a second portion including a first boss 211a and a second boss 211b, as shown in FIG. 3C. In some cases, the insulator rings 220 may not include the first boss 211a or the second boss 211b, but rather, may be a disk-like shape. In some cases, as shown in FIGS. 3A to 3D, the first boss 213a and the second boss 213b may extend away from a longitudinal axis of the central aperture 217. In some cases, an internal diameter of the conductive rings 200 and an internal diameter of the insulator rings 220 may be the same. In some cases, the internal diameter of the conductive rings 200 and an internal diameter of the insulator rings 220 may not be the same. In some cases, the conductive rings 200 and the insulator rings 220 may be stacked and interconnected using a core wire to hold the rings 200, 220 concentric, and the central apertures 215 and 217 align to form a connector lumen 216 that is open at a first end 201. The conductive rings 200 and the insulator rings 220 may be self-centering when stacked to form connector stack 250. When the conductive rings 200 and the insulator rings 220 are stacked and interconnected, the interconnected rings 200, 220 form an overlapping counterbore joint 240, as seen in FIG. 3D. The counterbore joint 240 may include an overlap within a range of about 20 to 50 percent of the length of the conductive rings 200.

In some cases, the interleaved conductive rings 200 and insulator rings 220 may be interconnected via brazing. For example, as shown in FIG. 3D, a braze material 226 is positioned between adjacent conductive rings 200 and insulator rings 220. The braze material 226 may be formed from, for example, gold, aluminum, copper, silver, magnesium, or the like. The braze material 226 may be positioned within the connector stack 250 between the conductive rings 200 and the insulator rings 220 as they are stacked. The braze material 226 may come in the form of a thin washer. When the conductive rings 200 and the insulator rings 220 are stacked and interconnected, the entire connector stack 250 is heated to its brazing temperature such that the braze material 226 liquefies, and the braze material 226 flows via capillary action within the spaces between the conductive rings 200 and the insulator rings 220, thereby bonding the rings 200, 220 together at a braze joint 225. The counterbore joints of the connector stack 250 allow the braze joint(s) 225 to be positioned at an angle parallel to the longitudinal axis of the connector stack 250. The structure of the counterbore joints provide additional resistance to shear (side or lateral) forces applied to the connector stack 250. In some cases, the interconnected conductive rings 200 and insulator rings 220 may be interconnected using any other suitable technique including, for example, 3D printing, co-firing (low or high temperature), or the like.

In some cases, an optional end piece 230 may be disposed along a second end 203 of the connector stack 250. The end piece 230 functions to close the second end 203 of the connector stack 250. In some cases, the end piece 230 is formed from an electrically nonconductive material. In some cases, the connector stack 250 including the end piece 230 may be interconnected to form a hermetic seal. An optional flange (not shown) can be coupled to the first end 201 of the connector stack 250 to facilitate insertion of an elongated member into the connector lumen 216, although this is not needed. A set screw assembly, spring, clip, or other securing element for holding an elongated member in the connector lumen 116 may be provided on either end of the connector stack.

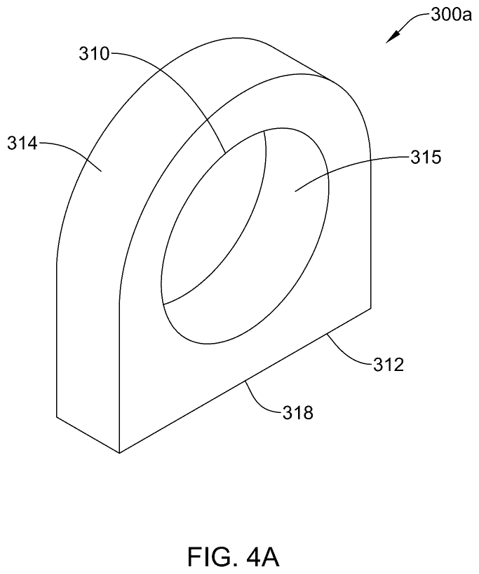

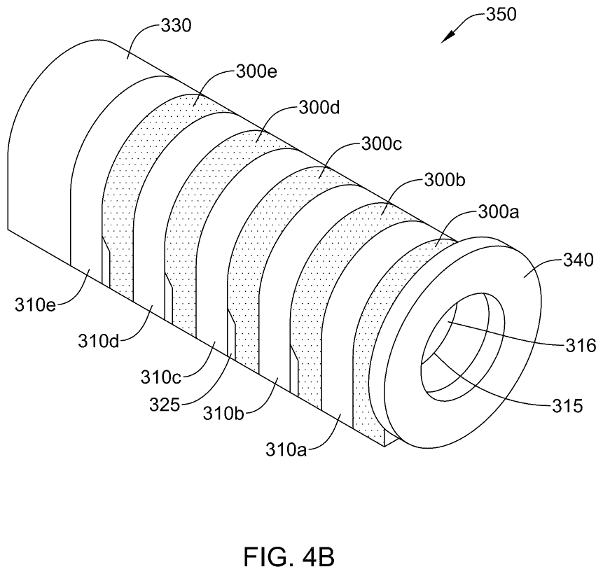

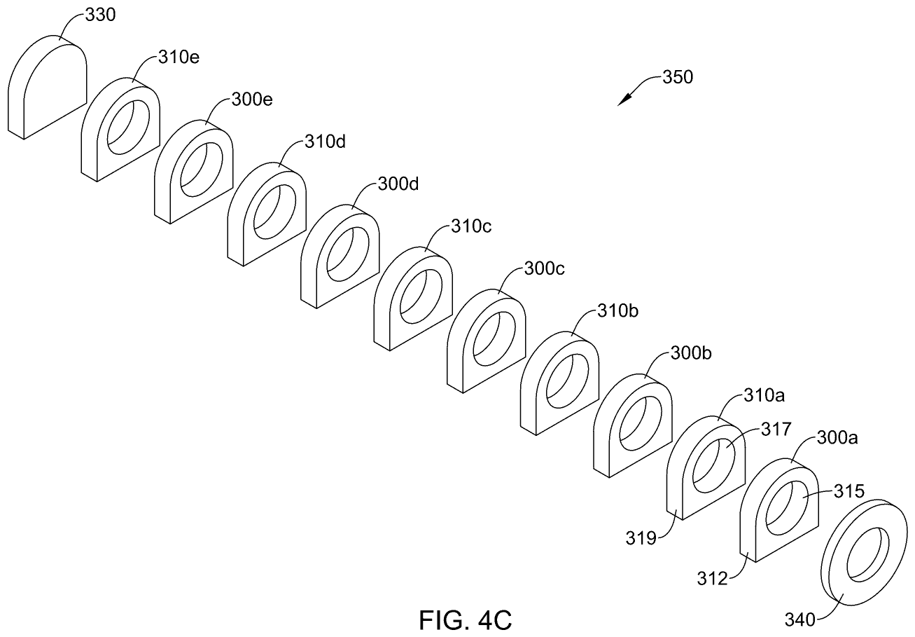

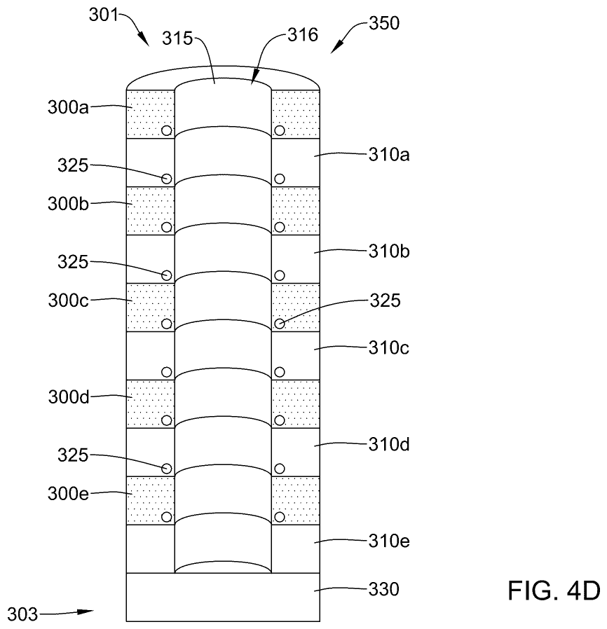

FIGS. 4A to 4D illustrate a connector shell, or connector stack 350, and the parts thereof. For example, FIG. 4A is a schematic perspective view of an illustrative conductive ring 300a. FIG. 4B is a schematic perspective view of an illustrative connector stack 350, including the conductive ring 300a as in FIG. 4A. FIG. 4C is a schematic, perspective exploded view of the illustrative connector stack 350 as in FIG. 4B, and FIG. 4D is a schematic, longitudinal cross-sectional view of the illustrative connector stack 350 as in FIG. 4B. The connector stack 350 may be configured to be used with the IMD 10 illustrated in FIG. 1, or the like. In such cases, the connector stack 350 may replace the connector stack assembly 20.

FIG. 4A illustrates a conductive ring 300a. The conductive ring 300a may be a section of the connector stack 350. The conductive ring 300a may be formed from an electrically conductive material, such as, for example, titanium, stainless steel (MP35N, for example), or other biocompatible and conductive metals. The conductive ring 300a may be formed via casting, working, machining, or the like, and then ground and/or polished as needed. The conductive ring 300a may include a shape that may include a first portion 310 having a flat disk shape with an inner, central aperture 315, and a second portion 318 including a straight edge 312, thereby giving the conductive ring 300a a D-flat profile.

The conductive ring 300a may be one of a plurality of conductive rings 300a, 300b, 300c, 300d, generally referred to herein as conductive rings 300. As shown in FIG. 4B, the conductive rings 300 may be interconnected with sections of electrically nonconductive material fixedly attached together. In some cases, the interconnected sections of material are stacked along a longitudinal length of the connector stack 350. The connector stack 350 may be configured to receive an elongated device (e.g., a lead, a lead extension, or other intermediary device). In some case, the interconnected sections of material are arranged in an alternating conductive-nonconductive configuration. In some cases, the interconnected sections of material are formed as alternating rings of material. For example, the connector stack 350 may be formed from alternating conductive rings 300, and a plurality of insulator rings 320a, 320b, 320c, 320d, 320c, generally referred to herein as insulator rings 320. The conductive rings 300 may be arranged such that each of the plurality of conductive rings 300 are separated from another one of the plurality of conductive rings 300 by at least one insulator ring 320 in either direction along the longitudinal length of the connector stack 350.

The insulator rings 320 may be formed from a nonconductive material such as, for example, glass, ceramic, or the like. The insulator rings 320 may be formed via sintering, molding, or the like. The insulator rings 320 may include a size and shape similar to that of the conductive rings 300. For example, the insulator rings 320 may include a shape that may include a first portion having a flat disk shape with an inner, central aperture 317, and a second portion including a straight edge 319. In some cases, an internal diameter of the conductive rings 300 and an internal diameter of the insulator rings 320 may be the same. In some cases, the internal diameter of the conductive rings 300 and an internal diameter of the insulator rings 320 may not be the same. In some cases, the conductive rings 300 and the insulator rings 320 may be stacked and interconnected using a core wire to hold the rings 300, 320 concentric, therefore the central apertures 315 and 317 align to collectively form a connector lumen 316 that is open at a first end 301 off the connector stack 350. In some cases, the conductive rings 300 and the insulator rings 320 are self-centering when stacked together to form the connector stack 350. When the conductive rings 300 and the insulator rings 320 align, the straight edges 312, 319 align to give the connector stack a D-flat profile. In such cases, the connector stack 350 is able to be positioned directly onto a PCBA (printed circuit board assembly). This would allow the elimination of a header and/or feedthrough structures.

In some cases, the interconnected conductive rings 300 and insulator rings 320 may be interconnected via brazing. For example, as shown in FIG. 4D, a braze material 326 is positioned between adjacent conductive rings 300 and insulator rings 320. The braze material 326 may be formed from, for example, gold, aluminum, copper, silver, magnesium, or the like. The braze material 326 may be positioned within the connector stack 350 between the conductive rings 300 and the insulator rings 320 as they are stacked. The braze material 326 may come in the form of a thin washer. When the conductive rings 300 and the insulator rings 320 are stacked and interconnected, the entire connector stack 350 is heated to its brazing temperature such that the braze material 326 liquefies, and the braze material 326 flows via capillary action within the spaces between the conductive rings 300 and the insulator rings 320, thereby bonding the rings 300, 320 together at a braze joint 325. The excess brazing material 326 may further flow toward the straight edges 312, 319 and couple the connector stack 350 to a PCBA. In some cases, the interconnected conductive rings 300 and insulator rings 320 may be interconnected using any other suitable technique including, for example, 3D printing, co-firing (low or high temperature), or the like.

In some cases, an optional end piece 330 may be disposed along a second end 303 of the connector stack 350. The end piece 330 functions to close the second end 303 of the connector stack 350. In some cases, the end piece 330 is formed from an electrically nonconductive material. In some cases, the connector stack 350 including the end piece 330 may be interconnected to form a hermetic seal. A set screw assembly, spring, clip, or other securing element for holding an elongated member in the connector lumen 116 may be provided on either end of the connector stack.

A flange 340 may be coupled to the first end 301 of the connector stack 350 to facilitate insertion of an elongated member into the connector lumen 316. A flange, such as flange 340, may be included at the open end of the designs of FIGS. 2A-2D, 3A-3D, and/or 5, if desired. For example, the flange 340 may be used in a manner as described in US PG Pub. 2023/0056675, the disclosure of which is incorporated herein by reference, to seal the assembled connector stack against fluid passing therethrough.

The illustrative example of FIGS. 4A-4D is presented using disk-shaped, flat conductive rings 300 and insulator rings 320. In other examples, the Chevron or conical shape version shown in FIGS. 2A-2D may be used rather than flat conductive rings 300 and insulator rings 320, while retaining the D-flat surface mount on one side. In other examples, the counterbore version shown in FIGS. 3A-3D may be used, rather than flat conductive rings 300 and insulator rings 320, while retaining the D-flat surface mount on one side.

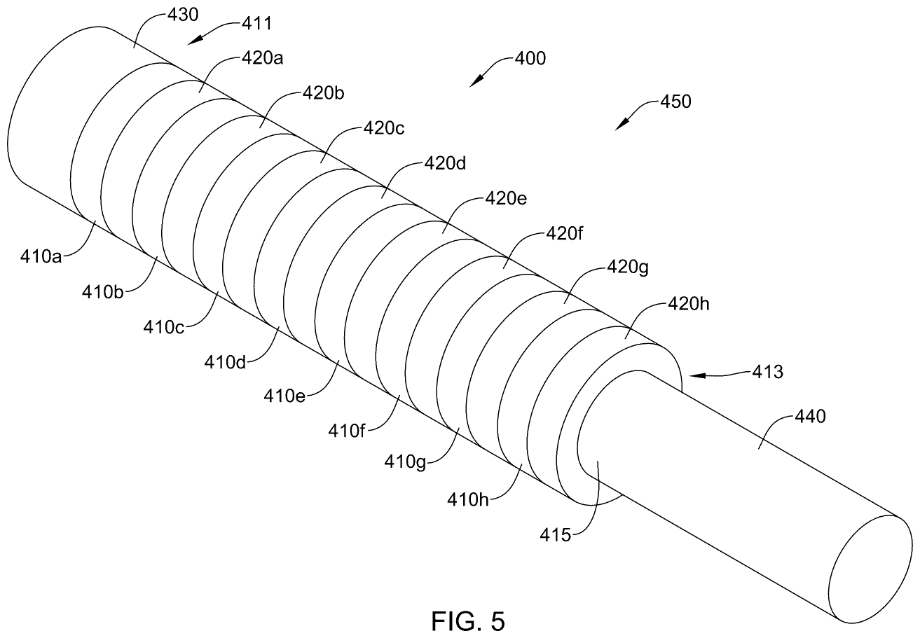

FIG. 5 is a schematic, perspective view of a portion of an elongated member 440 disposed in an illustrative connector stack 450. The connector stack 450 may be an example of any of the aforementioned connector stacks 150, 250, 350. The elongated member 440 may be a leadless cylindrical element, a lead, a lead extension or the like. The connector stack 450 may considered to be a connector shell having a first end 411 and an opposing second end 413. The first end may include an end piece 430. In some cases, the connector stack 450 and the end piece 430 may be interconnected to form a hermetic seal along the longitudinal length of the connector stack 450. The connector stack 450 may be formed from ring-shaped sections of electrically conductive material, or conductive rings 410a, 410b, 410c, 410d, 410e, 410f, 410g, 410h, generally referenced herein as conductive rings 410. The conductive rings 410 may be arranged in alternating configuration with ring-shaped sections of electrically non-conductive material, or insulator rings 420a, 520b, 420c, 420d, 420e, 420f, 420g, 420h, generally referenced herein as insulator rings 420. In this way, the conductive rings 410 are physically and electrically isolated from one another via the insulator rings 420.

The alternating conductive rings 410 and insulator rings 420 may be aligned along the longitudinal length of the connector stack 450. The conductive rings 410 may be electrically coupled to connector contacts (not explicitly shown), to which they are longitudinally aligned. The insulator rings 420 may be aligned along the longitudinal length of the connector stack 450 with spacers (not explicitly shown).

In at least some embodiments, one or more interconnect apertures are defined along sidewalls of the connector stack 450 to facilitate electrical coupling of interconnect conductors to the connector contacts. In some cases, interconnect apertures are defined along sidewalls of the conductor rings 410. The interconnect apertures may be suitable for enabling interconnect conductors (not shown in FIG. 5) to electrically couple with the connector contacts.

In other examples, interconnect conductors are electrically coupleable to connector contacts coupling the interconnect conductors to a bore-facing surface of the conductor rings 410 aligned with, and electrically coupled to, the connector contacts. For example, an interconnect conductor may be electrically coupled to the connector contact by electrically coupling the interconnect wire to a cavity-facing surface of the conductor rings 410, which, in turn, is electrically coupled to the connector contact. In such cases, the interconnect conductor extends entirely within the sealed cavity of an IMD (e.g., IMD 10 of FIG. 1).

In some cases, the elongated member 440 may be inserted into a lumen within the connector stack 450 and may be secured into place within the lumen of the connector stack 450 via, for example, welding, laser welding, soldering, or the like. The connector stack 450 and the elongated member 440 may be hermetically sealed using a hermetic sealing material including, for example, metal (e.g., solder, cermet, or the like), glass, or ceramic. In some cases, the elongated member 440 or embodiments with electrically conductive sealing material (e.g., vias), one or more of the conductive rings 410, conductors, etc. may be coated with one or more materials in preparation for accepting the hermetic sealing material. In such examples, the elongated member 440 and the connector stack 450 may stimulate outside of the lumen and serve as a micro-stimulator.

While it is shown that the conductive rings 410 and the insulator rings 420 include a ring-like shape, or cylindrical shape, other shapes may be contemplated, such as, for example, a D-flat shape, a cubical shape, a pyramidal shape, an oblong shape, or any other shape as desired. The conductive rings 410 and/or 420 may instead or in addition use the chevron design discussed above in relation to FIGS. 2A-2D, or the counterbore design discussed above in relation to FIGS. 3A-3D, with or without the D-flat shape of FIGS. 4A-4D.

In the preceding illustrative examples, the connector rings may or may not be the contacting members for the proximal end of a lead inserted into the connector stack. In some examples, additional coil contacts and seals can be provided within the bore defined by the connector stack to make direct contact with the electrical connectors on the lead.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A connector stack, comprising:
a plurality of conductive rings; and
a plurality of insulator rings, the plurality of insulator rings separating the conductive rings from each other, the plurality of conductive rings and the plurality of insulator rings forming the connector stack defining a lumen therethrough;
wherein each of the plurality of conductive rings each including a first boss.

2. The connector stack of claim 1, further comprising a braze material disposed between adjacent ones of each of the plurality of conductive rings and each of the plurality of insulator rings, wherein the plurality of conductive rings and the plurality of insulator rings are brazed together.

3. The connector stack of claim 1, wherein each of the plurality of insulator rings includes a second boss, and each of the plurality of conductive rings and each of the plurality of insulator rings are configured to stack together thereby forming overlapping counterbore joints.

4. The connector stack of claim 3, wherein the counterbore joints overlap within a range of about 20 to 50 percent.

5. The connector stack of claim 3, wherein the counterbore joints provide structural support against lateral displacement of the connector stack.

6. An implantable medical device comprising a port for receiving an implantable lead, the port comprising a connector stack as in claim 1.

7. A connector stack, comprising:
a plurality of conductive rings;
a plurality of insulator rings separating the conductive rings from each other, the plurality of conductive rings and the plurality of insulator rings forming the connector stack defining a lumen therethrough; and
a braze material between adjacent ones of the conductive rings and insulator rings;
wherein the plurality of conductive rings and the plurality of insulator rings are brazed together such that the connector stack is a hermetically sealed stack; and
wherein the connector stack includes a D-flat profile formed by an outer profile of the conductive rings having a D-flat shape.

8. The connector stack of claim 7, wherein the plurality of insulator rings are ceramic.

9. The connector stack of claim 7, wherein the connector stack is brazed together when the braze material flows via capillary action to a braze joint between adjacent ones of the conductive rings and insulator rings.

10. The connector stack of claim 7, wherein the connector stack includes a D-flat surface mount coupled to a proximal end or a distal end of the connector stack.

11. The connector stack of claim 7, wherein each of the plurality of conductive rings include a conical shape.

12. The connector stack of claim 11, wherein each of the plurality of insulator rings include a conical shape, and each of the plurality of conductive rings and each of the plurality of insulator rings are configured to stack together thereby forming chevron-shaped joints.

13. The connector stack of claim 7, wherein the plurality of conductive rings and the plurality of insulator rings are self-centering when stacked together.

14. The connector stack of claim 7, wherein each of the plurality of conductive rings includes a first boss.

15. The connector stack of claim 14, wherein each of the plurality of insulator rings includes a second boss, and each of the plurality of conductive rings and each of the plurality of insulator rings are configured to stack together thereby forming overlapping counterbore joints.

16. The connector stack of claim 15, wherein the counterbore joints overlap within a range of about 20 to 50 percent.

17. An implantable medical device comprising a port for receiving an implantable lead, the port comprising a connector stack as in claim 7.

18. A connector stack, comprising:
a plurality of conductive rings; and
a plurality of insulator rings, the plurality of insulator rings separating the conductive rings from each other, the plurality of conductive rings and the plurality of insulator rings forming the connector stack and defining a lumen therethrough;
wherein each of the plurality of conductive rings each include a conical shape.

19. The connector stack of claim 18, wherein each of the plurality of insulator rings include a conical shape, and each of the plurality of conductive rings and each of the plurality of insulator rings are configured to stack together thereby forming chevron-shaped joints.

20. The connector stack of claim 18, further comprising a braze material disposed between adjacent ones of each of the plurality of conductive rings and each of the plurality of insulator rings, wherein the plurality of conductive rings and the plurality of insulator rings are brazed together.
